(19) United States
(12) Patent Application Publication
(10) Pub. No.: US 2023/0015949 A1
(43) Pub. Date: Jan. 19, 2023

(54) NEISSERIA TREATMENT USING CANNABINOIDS

(71) Applicant: BOTANIX PHARMACEUTICALS LIMITED, North Perth, Western Australia (AU)

(72) Inventors: Matthew Callahan, Philadelphia, PA (US); Michael Thurn, Kangaroobie, (AU)

(21) Appl. No.: 17/756,536

(22) PCT Filed: Nov. 27, 2020
(86) PCT No.: PCT/AU2020/051284

§ 371 Date: May 26, 2022

(30) Foreign Application Priority Data
Nov. 29, 2019 (AU) 2019904528

(51) Int. Cl. A61K 36/185; A61K 31/352; A61P 31/00

(52) U.S. Cl. CPC A61K 36/185; A61K 31/352; A61P 31/00

(57) ABSTRACT

The use of a composition comprising a cannabinoid, an exemplary cannabinoid is cannabidiol, in the treatment or prevention of an infection by a Neisseria bacterium such as Neisseria gonorrhoea or Neisseria meningitidis.

### TECHNICAL FIELD

The present invention relates to a composition for the treatment or prevention of bacterial infections, particularly Neisseria infections, comprising a cannabinoid, and a method for use thereof.

### BACKGROUND ART

Compounds with antimicrobial properties have attracted great interest in recent times as a result of an increase in the prevalence of infections caused by bacteria, resulting in serious or fatal diseases. Furthermore, the regular use of broad spectrum antibiotic formulas has led to the increased occurrence of bacterial strains resistant to some antimicrobial compositions.

Novel antimicrobial compounds have the potential to be highly effective against these types of treatment-resistant bacteria. The pathogens, having not previously been exposed to the antimicrobial composition, may have little to no resistance to the treatment.

There is no indication that bacterial resistance to antibiotics will stop and for this reason new antibiotics and new treatment options are necessary to achieve a desirable treatment outcome in patients.

Neisseria bacteria cause serious infections and are increasingly resistant to multiple drugs and, in some cases, most available antibiotics.

There is a need to provide new methods for the treatment of infections by bacteria, particularly bacteria resistant to the current antibiotic compounds available and particularly Neisseria bacteria resistant to the current antibiotic compounds available. This invention seeks to provide such alternative treatment methods.

The previous discussion of the background art is intended to facilitate an understanding of the present invention only. The discussion is not an acknowledgement or admission that any of the material referred to is or was part of the common general knowledge as at the priority date of the application.

### SUMMARY OF INVENTION

The present invention provides a composition comprising a cannabinoid. Preferably the composition may be used for the treatment or prevention of an infection by a Neisseria bacterium. The present invention therefore provides a composition comprising a cannabinoid for the treatment or prevention of an infection by a Neisseria bacterium. The invention further provides a composition comprising cannabidiol and/or acids thereof for the treatment or prevention of an infection by a Neisseria bacterium.

According to another aspect of the invention, there is provided a method for the treatment or prevention of an infection by a Neisseria bacterium in a subject in need of such treatment comprising the step of:

administering an effective amount of a composition comprising a cannabinoid.

The invention further provides a method for the treatment or prevention of an infection by a Neisseria bacterium in a subject in need of such treatment comprising the step of:

administering an effective amount of a composition comprising cannabidiol and/or acids thereof.

The composition may be administered topically, orally, by injection, or by nasal or pulmonary administration.

According to another aspect of the invention, there is provided the use of a composition comprising a cannabinoid for the treatment or prevention of a Neisseria bacterial infection in a subject in need of such treatment or prevention. The invention further provides the use of a composition comprising cannabidiol and/or acids thereof for the treatment or prevention of a Neisseria bacterial infection in a subject in need of such treatment or prevention.

According to another aspect of the invention, there is provided the use of a cannabinoid, in the manufacture of composition for the treatment of an infection by a Neisseria bacterium in a subject. The invention further provides the use of cannabidiol and/or acids thereof in the manufacture of composition for the treatment of an infection by a Neisseria bacterium in a subject.

According to another aspect of the invention, there is provided a kit comprising a cannabinoid for the treatment or prevention of a Neisseria bacterial infection in a subject in need of such treatment or prevention. The invention further provides a kit comprising cannabidiol and/or acids thereof for the treatment or prevention of a Neisseria bacterial infection in a subject in need of such treatment or prevention.

Preferably, the cannabinoid is chosen from the list comprising: cannabidiol, cannabinol, cannabigerol, cannabichromene, and Δ⁹-tetrahydrocannabinol. Preferably, the cannabinoid is chosen from the list comprising: cannabidiol, cannabinolic acid. Most preferably, the cannabinoid is cannabidiol.

Preferably the Neisseria bacterial infection to be treated or prevented is caused by Neisseria gonorrhoea or Neisseria meningitidis.

### BRIEF DESCRIPTION OF THE DRAWINGS

Further features of the present invention are more fully described in the following description of several non-limiting embodiments thereof. This description is included solely for the purposes of exemplifying the present invention. It should not be understood as a restriction on the broad summary, disclosure or description of the invention as set out above. The description will be made with reference to the accompanying drawings in which:

FIGS. 1A and 1B are graphs of the activity of the MIC distributions of CBD and the comparators azithromycin, ceftriaxone and ciprofloxacin against 30 isolates of Neisseria gonorrhoeae. DESCRIPTION OF INVENTION

### DETAILED DESCRIPTION OF THE INVENTION

### Cannabinoid

The term cannabinoid includes compounds which interact with the cannabinoid receptor and various cannabinoid mimetics, such as certain tetrahydropyran analogs (e.g., Δ⁹-tetrahydrocannabinol, Δ⁸-tetrahydro-cannabinol, 6,6,9-trimethyl-3-pentyl-6H-dibenzo[b,d]pyran-1-ol, 3-(1,1-dimethylheptyl)-6,6a,7,8,10,10a-hexahydro-1-hydroxy-6,6-dimethyl-9H-dibenzo[b,d]pyran-9-one, (−)-(3S,4S)-7-hydroxy-Δ6-tetrahydrocannabinol-1,1-dimethylheptyl,(+)-(3S,4S)-7-hydroxy-Δ6-tetrahydrocannabinol-1,1-dimethylheptyl, 11-hydroxy-Δ⁹-tetrahydrocannabinol, and Δ8-tetrahydrocannabinol-11-oic acid)); certain piperidine analogs (e.g., (−)-(6S,6aR,9R,10aR)-5,6,6a,7,8,9,10,10a-octahydro-6-methyl-3-[(R)-1-methyl-4-phenylbutoxy]-1,9-phenanthridinediol-1-acetate)); certain aminoalkylindole analogs (e.g., (R)-(+)-[2,3-dihydro-5-methyl-3-(-4-morpholinylmethyl)-pyrrolo[1,2,3-de]-1,4-benzoxazin-6-yl]-1-naphthalenyl-methanone); and certain open pyran ring analogs (e.g., 2-[3-methyl-6-(1-methylethenyl)-2-cyclohexen-1-yl]-5-pentyl-1,3-benzenediol and 4-(1,1-dimethylheptyI)-2,3′-dihydroxy-6′alpha-(3-hydroxypropyl)-1′,2′,3′,4′,5′,6′-hexahydrobiphenyl).

Cannabinoids contemplated by the present invention include:
Cannabidiol (such as 2-[(1 S,6S)-3-methyl-6-prop-1-en-2-ylcyclohex-2-en-1-yl]-5-pentylbenzene-1,3-diol) and acids thereof (such as cannabidiolic acid—2,4-dihydroxy-3-[(1R,6R)-3-methyl-6-prop-1-en-2-ylcyclohex-2-en-1-yl]-6-pentylbenzoic acid);
Cannabinol (6,6,9-trimethyl-3-pentylbenzo[c]chromen-1-ol) and acids thereof (such as cannabinolic acid—1-hydroxy-6,6,9-trimethyl-3-pentylbenzo[c]chromene-2-carboxylic acid);
Δ⁹-tetrahydrocannabinol (such as (−)-Δ9-tetrahydrocannabinol) and acids thereof (such as Δ⁹-tetrahydrocannabinolic acid A—(6aR,10aR)-1-hydroxy-6,6,9-trimethyl-3-pentyl-6a,7,8,10a-tetrahydrobenzo[c]chromene-2-carboxylic acid);
11-hydroxy-Δ9-tetrahydrocannabinol (such as (±)-11-hydroxy-Δ09-tetrahydrocannabinol);
Cannabigerol (2-[(2E)-3,7-dimethylocta-2,6-dienyl]-5-pentylbenzene-1,3-diol) and acids thereof (such as cannabigerolic acid—3-[(2E)-3,7-dimethylocta-2,6-dienyl]-2,4-dihydroxy-6-pentylbenzoic acid);
Tetrahydrocannabivarin—(such as (6aR,10aR)-6,6,9-trimethyl-3-propyl-6a,7,8,10a-tetrahydrobenzo[c]chromen-1-ol) and acids thereof (such as tetrahydrocannabivarinic acid —(6aR,10aR)-1-hydroxy-6,6,9-trimethyl-3-propyl-6a,7,8,10a-tetrahydrobenzo[c]chromene-2-carboxylic acid);
Cannabichromene (2-methyl-2-(4-methylpent-3-enyl)-7-pentylchromen-5-ol);
Nantradol hydrochloride ((−)-(6S,6aR,9R,10aR)-5,6,6a,7,8,9,10,10a-octahydro-6-methyl-3-[(R)-1-methyl-4-phenylbutoxy]-1,9-phenanthridinediol-1-acetate);
Nabilone (3-(1,1-dimethylheptyl)-6,6a,7,8,10,10a-hexahydro-1-hydroxy-6,6-dimethyl-9H-dibenzo[b,d]pyran-9-one);
6,6,9-trimethyl-3-pentyl-6H-dibenzo[b,d]pyran-1-ol (such as (−)-Δ8-tetrahydrocannabinol);
(3S,4S)-7-hydroxy-Δ6-tetrahydrocannabinol-1,1-dimethylheptyl (such as (−)-(3S,4S)-7-hydroxy-Δ6-tetrahydrocannabinol-1,1-dimethylheptyl and (+)-(3S,4S)-7-hydroxy-Δ6-tetrahydrocannabinol-1,1-dimethylheptyl);
Δ8-tetrahydrocannabinol-11-oic acid ((6aR,10aR)-1-hydroxy-6,6-dimethyl-3-pentyl-6a,7,10,10a-tetrahydrobenzo[c]chromene-9-carboxylic acid);
(R)-(+)-[2,3-dihydro-5-methyl-3-(-4-morpholinylmethyl)-pyrrolo[1,2,3-de]-1,4-benzoxazin-6-yl]-1-naphthalenyl-methanone;
4-(1,1-dimethylheptyl)-2,3′-dihydroxy-6′alpha-(3-hydroxypropyl)-1′,2′,3′,4′,5′,6′-hexahydrobiphenyl;
(6aR,10aR)-9-(hydroxymethyl)-6,6-dimethyl-3-pentyl-6a,7,8,10a-tetrahydrobenzo[c]chromen-1-ol.

### Abbreviations

### Cannbidiol—CBD

Cannabidiolic acid—CBDA

### Cannabinol—CBN

Cannabinolic acid—CBNA
Δ⁹-tetrahydrocannabinol—THC
Δ⁹-tetrahydrocannabinolic acid A—THCA-A

### Cannabigerol—CBG

Cannabigerolic acid—CBGA

### Cannabichromene—CBC

### Tetrahydrocannabivarin—THCV

### Tetrahydrocannabivarinic Acid—THCVA

Preferably, the cannabinoid is chosen from the list comprising: cannabidiol, cannabinol, cannabinolic acid, cannabigerol, cannabichromene, and Δ⁹-tetrahydrocannabinol. Preferably, the cannabinoid is chosen from the list comprising: cannabidiol, cannabinolic acid. Most preferably, the cannabinoid is cannabidiol.

Cannabidiol, as used herein, refers to 2-[3-methyl-6-(1-methylethenyl)-2-cyclohexen-1-yl]-5-pentyl-1,3-benzenediol. The synthesis of cannabidiol is described, for example, in Petilka et al., Helv. Chim. Acta, 52: 1102 (1969) and in Mechoulam et al., J. Am. Chem. Soc., 87:3273 (1965), which are hereby incorporated by reference.

### Composition

According to one aspect of the invention, there is provided a composition comprising a cannabinoid. Preferably the composition may be used for the treatment or prevention of an infection by a Neisseria bacterium. There is therefore provided a composition comprising a cannabinoid for use in the treatment or prevention of an infection by a Neisseria bacterium. The invention further provides a composition comprising cannabidiol and/or acids thereof for the treatment or prevention of an infection by a Neisseria bacterium.

Preferably, the cannabinoid is chosen from the list comprising: cannabidiol, cannabinol, cannabigerol, cannabichromene, and Δ⁹-tetrahydrocannabinol. Preferably, the cannabinoid is chosen from the list comprising: cannabidiol, cannabinolic acid. Most preferably, the cannabinoid is cannabidiol.

The invention therefore provides a composition comprising cannabidiol and/or acids thereof for the treatment or prevention of an infection by a Neisseria bacterium.

Preferably the Neisseria bacterial infection to be treated or prevented is caused by Neisseria gonorrhoea or Neisseria meningitidis.

The compositions may contain more than one cannabinoid. For example, the composition of the present invention may contain a combination of two, three or more cannabinoids. In preferred forms, at least one such cannabinoid is cannabidiol.

The term “infection” as used herein means colonization by a micro-organism and/or multiplication of a micro-organism, in particular, a bacterium and more particularly a Neisseria bacterium. The infection may be unapparent or result in local cellular injury. The infection may be localized, subclinical and temporary or alternatively may spread by extension to become an acute or chronic clinical infection. The infection may also be a latent infection, in which the microorganism is present in a subject, however the subject does not exhibit symptoms of disease associated with the organism.

Preferably the composition of the present invention delivers a therapeutically effective amount of the cannabinoid to the subject.

The phrase “therapeutically effective amount” as used herein refers to an amount of the cannabinoid sufficient to inhibit bacterial growth associated with bacterial carriage or a bacterial infection. That is, reference to the administration of the therapeutically effective amount of a cannabinoid according to the methods or compositions of the invention refers to a therapeutic effect in which substantial bacteriocidal or bacteriostatic activity causes a substantial inhibition of the bacterial carriage or bacterial infection. The term “therapeutically effective amount” as used herein, refers to a nontoxic but sufficient amount of the composition to provide the desired biological, therapeutic, and/or prophylactic result. The desired results include elimination of bacterial carriage or reduction and/or alleviation of the signs, symptoms, or causes of a disease, or any other desired alteration of a biological system. An effective amount in any individual case may be determined by one of ordinary skill in the art using routine experimentation. In relation to a pharmaceutical composition, effective amounts can be dosages that are recommended in the modulation of a diseased state or signs or symptoms thereof. Effective amounts differ depending on the pharmaceutical composition used and the route of administration employed. Effective amounts are routinely optimized taking into consideration various factors of a particular patient, such as age, weight, gender, etc and the area affected by disease or disease-causing microorganisms.

As used herein, “treating” or “treatment” refers to inhibiting the disease or condition, i.e., arresting or reducing its development or at least one clinical or subclinical symptom thereof, for example reducing or eliminating a bacterial infection. “Treating” or “treatment” further refers to relieving the disease or condition, i.e., causing regression of the disease or condition or at least one of its clinical or subclinical symptoms. The benefit to a subject to be treated is either statistically significant or at least perceptible to the subject and/or the physician. In the context of treating a bacterial infection, the term treatment includes reducing or eliminating colonization by bacteria and/or multiplication of bacteria, preferably Neisseria bacteria.

In one form of the invention, reducing or eliminating colonization by bacteria means reducing or eliminating colonization by bacteria as measured by % bacteria killed.

In one form of the invention, reducing or eliminating colonization by bacteria means reducing or eliminating colonization by bacteria as measured by a log₁₀ reduction in bacteria.

Preferably the compositions of the present invention do not contain any additional compound that removes or substantially removes or reduces the integrity of the outer membrane of a Gram-negative bacteria.

### Method of Treatment

According to another aspect of the invention, there is provided a method for the treatment or prevention of an infection by a Neisseria bacterium in a subject in need of such treatment comprising the step of:

administering an effective amount of a composition comprising a cannabinoid.

Preferably, the cannabinoid is chosen from the list comprising: cannabidiol, cannabinol, cannabigerol, cannabichromene, and Δ⁹-tetrahydrocannabinol. Preferably, the cannabinoid is chosen from the list comprising: cannabidiol, cannabinolic acid. Most preferably, the cannabinoid is cannabidiol.

The invention therefore provides a method for the treatment or prevention of an infection by a Neisseria bacterium in a subject in need of such treatment comprising the step of:

administering an effective amount of a composition comprising cannabidiol and/or acids thereof.

Preferably the Neisseria bacterial infection to be treated or prevented is caused by Neisseria gonorrhoea or Neisseria meningitidis.

### Topical Infections

In one aspect, the composition used in the method for the treatment or prevention of an infection by a Neisseria bacterium in a subject in need of such treatment is a topical pharmaceutical composition for the treatment of an infection of a dermal or mucosal surface.

The method for topical treatment may comprise the administration of a cannabinoid directly to a dermal or mucosal surface of the subject. Preferably, the cannabinoid is applied topically to the skin or mucosal membranes (oral, vaginal, rectal) of the subject.

### Infections Treated by Oral Administration

In one aspect, the composition used in the method for the treatment or prevention of an infection by a Neisseria bacterium in a subject in need of such treatment is an oral pharmaceutical composition comprising a cannabinoid for the treatment of an infection. Any infection in a subject by a Neisseria bacterium may be treated using an orally administered treatment method.

The oral treatment method may comprise the administration of a cannabinoid to the gastrointestinal (GI) tract of the subject. Preferably, the cannabinoid enters the blood stream via absorption in the GI tract and is systemically available to the subject. However, the oral treatment method may comprise administering the cannabinoid to the GI tract for a localised effect.

### Infections Treated by Injection

In one aspect, the composition used in the method for the treatment or prevention of an infection by a Neisseria bacterium in a subject in need of such treatment is an injected pharmaceutical composition for the treatment of an infection. Any infection in a subject by a Neisseria bacteria may be treated using a method of treatment of injected cannabinoids.

The injection treatment method may be by intravenous injection, intramuscular injection, or intraperitoneal injection. The administration may be intraventricularly, intracranially, intracapsularly, intraspinally, or intracisternally. Preferably, the injection treatment method is by intravenous or intramuscular injection. Preferably, the cannabinoid enters the blood stream via IV administration or a subcutaneous bolus and is systemically available to the subject.

### Infections Treated by Nasal or Pulmonary Administration

In one aspect, the composition used in the method for the treatment or prevention of an infection by a Neisseria bacterium in a subject in need of such treatment is a nasal or pulmonary pharmaceutical composition for the treatment of an infection. Any infection in a subject by a bacteria may be treated using a nasal or pulmonary delivered treatment method.

Preferably, infections of the nasal cavity, sinuses, respiratory tract and lungs are treated using a nasal or pulmonary treatment method. The nasal or pulmonary treatment method may comprise the administration of a cannabinoid to the nasal or pulmonary system of the subject. The cannabinoid may enter the blood stream via absorption in the nasal or pulmonary system and be systemically available to the subject. However, the cannabinoid dosing method may alternatively comprise administering the cannabinoid to the nasal or pulmonary system for a localised effect.

### Additional Antimicrobials

Other active agents may also be incorporated into the composition of the present invention. For example, additional antimicrobial agents such as antibacterials, antifungals etc may be incorporated. In a preferred form of the invention, the additional antimicrobial agent is not a compound that removes or substantially removes or reduces the integrity of the outer membrane of a Gram-negative bacteria.

For example, the composition may further comprise benzoyl peroxide, erythromycin, clindamycin, doxycycline or meclocycline.

Additional antimicrobial agents that can be used include, but are not limited to silver compounds (e.g., silver chloride, silver nitrate, silver oxide), silver ions, silver particles, iodine, povidone/iodine, chlorhexidine, 2-p-sulfanilyanilinoethanol, 4,4′-sulfinyldianiline, 4-sulfanilamidosalicylic acid, acediasulfone, acetosulfone, amikacin, amoxicillin, amphotericin B, ampicillin, apalcillin, apicycline, apramycin, arbekacin, aspoxicillin, azidamfenicol, azithromycin, aztreonam, bacitracin, bambermycin(s), biapenem, brodimoprim, butirosin, capreomycin, carbenicillin, carbomycin, carumonam, cefadroxil, cefamandole, cefatrizine, cefbuperazone, cefclidin, cefdinir, cefditoren, cefepime, cefetamet, cefixime, cefinenoxime, cefminox, cefodizime, cefonicid, cefoperazone, ceforanide, cefotaxime, cefotetan, cefotiam, cefozopran, cefpimizole, cefpiramide, cefpirome, cefprozil, cefroxadine, ceftazidime, cefteram, ceftibuten, ceftriaxone, cefuzonam, cephalexin, cephaloglycin, cephalosporin C, cephradine, chloramphenicol, chlortetracycline, ciprofloxacin, clarithromycin, clinafloxacin, clindamycin, clomocycline, colistin, cyclacillin, dapsone, demeclocycline, diathymosulfone, dibekacin, dihydrostreptomycin, dirithromycin, doxycycline, enoxacin, enviomycin, epicillin, erythromycin, flomoxef, fortimicin(s), gentamicin(s), glucosulfone solasulfone, gramicidin S, gramicidin(s), grepafloxacin, guamecycline, hetacillin, imipenem, isepamicin, josamycin, kanamycin(s), leucomycin(s), lincomycin, lomefloxacin, lucensomycin, lymecycline, meclocycline, meropenem, methacycline, micronomicin, midecamycin(s), minocycline, moxalactam, mupirocin, nadifloxacin, natamycin, neomycin, netilmicin, norfloxacin, oleandomycin, oxytetracycline, p-sulfanilylbenzylamine, panipenem, paromomycin, pazufloxacin, penicillin N, pipacycline, pipemidic acid, polymyxin, primycin, quinacillin, ribostamycin, rifamide, rifampin, rifamycin SV, rifapentine, rifaximin, ristocetin, ritipenem, rokitamycin, rolitetracycline, rosaramycin, roxithromycin, salazosulfadimidine, sancycline, sisomicin, sparfloxacin, spectinomycin, spiramycin, streptomycin, succisulfone, sulfachrysoidine, sulfaloxic acid, sulfamidochrysoidine, sulfanilic acid, sulfoxone, teicoplanin, temafloxacin, temocillin, tetracycline, tetroxoprim, thiamphenicol, thiazolsulfone, thiostrepton, ticarcillin, tigemonam, tobramycin, tosufloxacin, trimethoprim, trospectomycin, trovafloxacin, tuberactinomycin, vancomycin, azaserine, candicidin(s), chlorphenesin, dermostatin(s), filipin, fungichromin, mepartricin, nystatin, oligomycin(s), ciproflaxacin, norfloxacin, ofloxacin, pefloxacin, enoxacin, rosoxacin, amifloxacin, fleroxacin, temafloaxcin, lomefloxacin, perimycin A or tubercidin, and the like.

### Subject

The subject may be any subject capable of infection by a bacterium, particularly a Neisseria bacteria. The subject may be mammalian or avian. Preferably, the subject is selected from the group comprising human, canine, avian, porcine, bovine, ovine, equine, and feline. More preferably, the subject is selected from the group comprising human, bovine, porcine, equine, feline and canine; most preferably human.

### Use

According to another aspect of the invention, there is provided the use of a composition comprising a cannabinoid for the treatment or prevention of a Neisseria bacterial infection in a subject in need of such treatment or prevention.

According to another aspect of the invention, there is provided the use of a cannabinoid, in the manufacture of composition for the treatment of an infection by a Neisseria bacterium in a subject.

Preferably, the cannabinoid is chosen from the list comprising: cannabidiol, cannabinol, cannabigerol, cannabichromene, and Δ⁹-tetrahydrocannabinol. Preferably, the cannabinoid is chosen from the list comprising: cannabidiol, cannabinolic acid. Most preferably, the cannabinoid is cannabidiol.

The invention therefore provides the use of a composition comprising cannabidiol and/or acids thereof for the treatment or prevention of a Neisseria bacterial infection in a subject in need of such treatment or prevention. The invention further provides the use of cannabidiol and/or acids thereof in the manufacture of composition for the treatment of an infection by a Neisseria bacterium in a subject.

Preferably the Neisseria bacterial infection to be treated or prevented is caused by Neisseria gonorrhoea or Neisseria meningitidis.

### Delivery

In one embodiment of the invention, the cannabinoid is administered to the subject using a dosing regimen selected from the group consisting of: three times daily; two times daily; daily; every second day, every third day, once weekly; once fortnightly and once monthly.

In accordance with certain embodiments, the composition is administered regularly until treatment is obtained. In one preferred embodiment, the composition is administered to the subject in need of such treatment using a dosing regimen selected from the group consisting of: every hour, every 2 hours, every 3 hours, once daily, twice daily, three times daily, four times daily, five times daily, once weekly, twice weekly, once fortnightly and once monthly. However, other application schedules may be utilized in accordance with the present invention. Preferably, the composition of the treatment regimen is administered to the subject between one and five times per day, more preferably once or twice per day.

The compositions used in the treatment methods of the invention may be administered by injection, or prepared for oral, inhaled (pulmonary), nasal, or any other form of administration. Preferably the compositions are administered, for example, orally, intravenously, subcutaneously, intramuscularly, intraventricularly, intracranially, intracapsularly, intraspinally, intracisternally, intraperitoneally, intranasally or by aerosol administration.

The mode of administration is preferably suitable for the form in which the composition has been prepared. The mode of administration for the most effective response may be determined empirically and the means of administration described below are given as examples, and do not limit the method of delivery of the composition of the present invention in any way. All the above compositions are commonly used in the pharmaceutical industry and are commonly known to suitably qualified practitioners.

The compositions of the invention may optionally include pharmaceutically acceptable nontoxic excipients and carriers. As used herein, a “pharmaceutical carrier” is a pharmaceutically acceptable solvent, suspending agent, excipient or vehicle for delivering the cannabinoid to the subject. The carrier may be liquid or solid, and is selected with the planned manner of administration in mind.

The composition of the invention may be selected from the group consisting of: an immediate release composition, a delayed release composition, a controlled release composition and a rapid release composition.

The composition of the invention may further comprise an anti-inflammatory agent (such as a corticosteroid). If the composition is a topical composition, an anticomedolyic agent (such as tretinoin), and/or a retinoid or derivative thereof may also be added.

The compositions described herein may be formulated by including such dosage forms in an oil-in-water emulsion, or a water-in-oil emulsion. In such a composition, the immediate release dosage form is in the continuous phase, and the delayed release dosage form is in a discontinuous phase. The composition may also be produced in a manner for delivery of three dosage forms as hereinabove described. For example, there may be provided an oil-in-water-in-oil emulsion, with oil being a continuous phase that contains the immediate release component, water dispersed in the oil containing a first delayed release dosage form, and oil dispersed in the water containing a third delayed release dosage form.

The compositions described herein may be in the form of a liquid composition. The liquid composition may comprise a solution that includes a therapeutic agent (e.g. a cannabinoid) dissolved in a solvent. Generally, any solvent that has the desired effect may be used in which the therapeutic agent dissolves and which can be administered to a subject. Generally, any concentration of therapeutic agent that has the desired effect can be used. The composition in some variations is a solution which is unsaturated, a saturated or a supersaturated solution. The solvent may be a pure solvent or may be a mixture of liquid solvent components. In some variations the solution formed is an in-situ gelling composition. Solvents and types of solutions that may be used are well known to those versed in such drug delivery technologies.

The composition may or may not contain water. Preferably, the composition does not contain water, i.e. it is non-aqueous. In another preferred embodiment, the composition does not comprise a preservative.

The administration of the cannabinoid in accordance with the methods and compositions of the invention may be by any suitable means that results in an amount sufficient to treat a microbial infection or to reduce microbial growth at the location of infection.

The cannabinoid may be contained in any appropriate amount and in any suitable carrier substance, and is generally present in an amount of 1-95% by weight of the total weight of the composition.

The pharmaceutical composition may be formulated according to the conventional pharmaceutical or veterinary practice (see, for example, Remington: The Science and Practice of Pharmacy, 20th edition, 2000, ed; A. R. Gennaro, Lippincott Williams & Wilkins, Philadelphia, and Encyclopedia of Pharmaceutical Technology, eds; J. Swarbrick and J. C. Boylan, 1988-1999, Marcel Dekker, New York; Remington's Pharmaceutical Sciences, 18^{th} Edition, Mack Publishing Company, Easton, Pa., USA).

Generally, examples of suitable carriers, excipients and diluents include, without limitation, water, saline, ethanol, dextrose, glycerol, lactose, dextrose, sucrose, sorbitol, mannitol, starches, gum acacia, calcium phosphates, alginate, tragacanth, gelatine, calcium silicate, microcrystalline cellulose, polyvinylpyrrolidone, cellulose, water syrup, methyl cellulose, methyl and propylhydroxybenzoates, polysorbates, talc magnesium stearate, mineral oil or combinations thereof. The compositions can additionally include lubricating agents, pH buffering agents, wetting agents, emulsifying and suspending agents, preserving agents, sweetening agents or flavouring agents.

The composition may be in the form of a controlled-release composition and may include a degradable or non-degradable polymer, hydrogel, organogel, or other physical construct that modifies the release of the cannabinoid. It is understood that such compositions may include additional inactive ingredients that are added to provide desirable colour, stability, buffering capacity, dispersion, or other known desirable features. Such compositions may further include liposomes, such as emulsions, foams, micelles, insoluble monolayers, liquid crystals, phospholipid dispersions, lamellar layers and the like. Liposomes for use in the invention may be formed from standard vesicle-forming lipids, generally including neutral and negatively charged phospholipids and a sterol, such as cholesterol.

### Topical Compositions

Compositions of the invention may be administered topically. Therefore, contemplated for use herein are compositions adapted for the direct application to the skin.

The composition may be in a form selected from the group comprising suspensions, emulsions, liquids, creams, oils, lotions, ointments, gels, hydrogels, pastes, plasters, roll-on liquids, skin patches, sprays, glass bead dressings, synthetic polymer dressings and solids. For instance, the compositions of the invention may be provided in the form of a water-based composition or ointment which is based on organic solvents such as oils. Alternatively, the compositions of the invention may be applied by way of a liquid spray comprising film forming components and at least a solvent in which the cannabinoid is dispersed or solubilised.

The composition of the invention may be provided in a form selected from the group comprising, but not limited to, a rinse, a shampoo, a lotion, a gel, a leave-on preparation, a wash-off preparation, and an ointment.

Various topical delivery systems may be appropriate for administering the compositions of the present invention depending up on the preferred treatment method. Topical compositions may be produced by dissolving or combining the cannabinoid in an aqueous or non-aqueous carrier. In general, any liquid, cream, or gel or similar substance that does not appreciably react with the cannabinoid or any other of the active ingredients that may be introduced into the composition and which is non-irritating is suitable. Appropriate non-sprayable viscous, semi-solid or solid forms can also be employed that include a carrier compatible with topical application and have dynamic viscosity preferably greater than water.

Suitable compositions are well known to those skilled in the art and include, but are not limited to, solutions, suspensions, emulsions, creams, gels, ointments, powders, liniments, salves, aerosols, transdermal patches, etc., which are, if desired, sterilised or mixed with auxiliary agents, e.g. preservatives, stabilisers, emulsifiers, wetting agents, fragrances, colouring agents, odour controllers, thickeners such as natural gums, etc. Particularly preferred topical compositions include ointments, creams or gels.

Ointments generally are prepared using either (1) an oleaginous base, i.e., one consisting of fixed oils or hydrocarbons, such as white petroleum, mineral oil, or (2) an absorbent base, i.e., one consisting of an anhydrous substance or substances which can absorb water, for example anhydrous lanolin. Customarily, following formation of the base, whether oleaginous or absorbent, the cannabinoids are added to an amount affording the desired concentration.

Creams are oil/water emulsions. They consist of an oil phase (internal phase), comprising typically fixed oils, hydrocarbons and the like, waxes, petroleum, mineral oil and the like and an aqueous phase (continuous phase), comprising water and any water-soluble substances, such as added salts. The two phases are stabilised by use of an emulsifying agent, for example, a surface active agent, such as sodium lauryl sulfite; hydrophilic colloids, such as acacia colloidal clays, veegum and the like. Upon formation of the emulsion, the cannabinoids can be added in an amount to achieve the desired concentration.

Gels comprise a base selected from an oleaginous base, water, or an emulsion-suspension base. To the base is added a gelling agent that forms a matrix in the base, increasing its viscosity. Examples of gelling agents are hydroxypropyl cellulose, acrylic acid polymers and the like. Customarily, the cannabinoid is added to the composition at the desired concentration at a point preceding addition of the gelling agent.

The amount of cannabinoid incorporated into a topical composition is not critical; the concentration should be within a range sufficient to permit ready application of the composition such that an effective amount of the cannabinoids is delivered.

### Oral Compositions

Compositions of the invention may be administered orally.

Contemplated for use herein are oral solid dosage forms, which are described generally in Martin, Remington's Pharmaceutical Sciences, 18th Ed. (1990 Mack Publishing Co. Easton Pa. 18042) at Chapter 89, which is herein incorporated by reference. Solid dosage forms include tablets, capsules, pills, troches or lozenges, cachets or pellets. Also, liposomal or proteinoid encapsulation may be used to formulate the present compositions (as, for example, proteinoid microspheres reported in U.S. Pat. No. 4,925,673). Liposomal encapsulation may be used and the liposomes may be derivatised with various polymers (E.g., U.S. Pat. No. 5,013,556). A description of possible solid dosage forms for the therapeutic is given by Marshall, in Modern Pharmaceutics, Chapter 10, Banker and Rhodes ed., (1979), herein incorporated by reference. In general, the composition will include therapeutic agents (e.g. a cannabinoid), and inert ingredients which allow for protection against the stomach environment, and release of the cannabinoid in the intestine.

For the cannabinoid of the invention, the location of release may be the stomach, the small intestine (the duodenum, the jejunum, or the ileum), or the large intestine. One skilled in the art has available compositions that will not dissolve in the stomach, yet will release the material in the duodenum or elsewhere in the intestine. In one aspect, the release will avoid the deleterious effects of the stomach environment, either by protection of the composition or by release of the cannabinoid beyond the stomach environment, such as in the intestine.

It is believed that the oral bioavailability of cannabinoids is only 4% to 12% and absorption is highly variable. Although most cannabinoids are generally easily absorbed due to their high partition coefficient (P), they are subject to degradation in the stomach and significant first-pass metabolism.

Preferably, the cannabinoid is released in the lower gastrointestinal tract.

The oral dosage method may be provided using an oral sustained release pharmaceutical composition comprising a therapeutically effective pharmaceutical composition according to the invention, and a release retardant.

In one aspect of the present invention the release retardant is a water-soluble, water swellable and/or water insoluble polymer. In particular, water-soluble polymers are selected from the group comprising are ethylcellulose, hydroxypropyl cellulose, hydroxypropyl methyl cellulose, an enteric coating; and a semipermeable membrane. In another aspect of the invention the release retardant is a non-polymeric release retardant. More particularly, the non-polymeric release retardant is hydrogenated castor oil. The compositions of the invention may be milled or granulated and compressed into tablets or encapsulated into capsules according to conventional procedures known in the art.

To ensure full gastric resistance, a coating impermeable to at least pH 5.0 is used. Examples of the more common inert ingredients that are used as enteric coatings are cellulose acetate trimellitate (CAT), hydroxypropylmethylcellulose phthalate (HPMCP), HPMCP 50, HPMCP 55, polyvinyl acetate phthalate (PVAP), Eudragit L30D, Aquateric, cellulose acetate phthalate (CAP), Eudragit L, Eudragit S, and Shellac. These coatings may be used as mixed films.

A coating or mixture of coatings can also be used on tablets, which are not intended for protection against the stomach. This includes without limitation sugar coatings, or coatings that make the tablet easier to swallow. Exemplary capsules consist of a hard shell (such as gelatin) for delivery of dry therapeutic i.e. powder; for liquid forms, a soft gelatine shell may be used. The shell material of cachets in certain aspects is thick starch or other edible paper. For pills, lozenges, moulded tablets or tablet triturates, moist massing techniques are also contemplated, without limitation.

As used herein, the term “sustained release” means the gradual but continuous or sustained release over a relatively extended period of the therapeutic compound content after oral ingestion. The release may continue after the pharmaceutical composition has passed from the stomach and through until and after the pharmaceutical composition reaches the intestine. The phrase “sustained release” also means delayed release wherein release of the therapeutic compound is not immediately initiated upon the pharmaceutical composition reaching the stomach but rather is delayed for a period of time, for example, until when the pharmaceutical composition reaches the intestine. Upon reaching the intestine, the increase in pH may then trigger release of the therapeutic agent from the pharmaceutical composition.

Though term “release retardant” is used herein, means a substance that reduces the rate of release of a therapeutic agent from a pharmaceutical composition when orally ingested. The release retardant may be a polymer or a non-polymer. The release retardant may be used according to any one of several sustained release systems including, for example, a diffusion system, a dissolution system and/or an osmotic system.

In certain aspects, the therapeutic agent (e.g. a cannabinoid) is included in the composition as fine multi-particulates in the form of granules or pellets of particle size about 1 mm. The composition of the material for capsule administration is, in certain aspects, a powder, lightly compressed plug, or even as a tablet. In one aspect, the composition comprising the therapeutic agent could be prepared by compression.

Colourants and flavouring agents may optionally be included. For example, compositions may be formulated (such as, and without limitation, by liposome or microsphere encapsulation) and then further contained within an edible product, such as a refrigerated beverage containing colorants and flavouring agents.

The volume of the composition may be diluted or increased with an inert material. These diluents could include carbohydrates, especially mannitol, alpha-lactose, anhydrous lactose, cellulose, sucrose, modified dextrans and starch. Certain inorganic salts are also optionally used as fillers including calcium triphosphate, magnesium carbonate and sodium chloride. Some commercially available diluents are Fast-Flo, Emdex, STA-Rx 1500, Emcompress and Avicell.

In other embodiments, disintegrants are included in the solid dosage form compositions of the present invention. Materials used as disintegrants include but are not limited to starch including the commercial disintegrant based on starch, Explotab. Sodium starch glycolate, Amberlite, sodium carboxymethylcellulose, ultramylopectin, sodium alginate, gelatine, orange peel, acid carboxymethyl cellulose, natural sponge and bentonite are also contemplated. Another form of the disintegrants is the insoluble cationic exchange resins. Powdered gums are also optionally used as disintegrants and as binders and these include, without limitation, powdered gums such as agar, Karaya or tragacanth. Alginic acid and its sodium salt are also useful as disintegrants.

Binders are contemplated to hold the cannabinoid together to form a hard tablet and include, without limitation, materials from natural products such as acacia, tragacanth, starch and gelatine. Other binders include, without limitation, methylcellulose (MC), ethyl cellulose (EC) and carboxymethyl cellulose (CMC). Polyvinyl pyrrolidone (PVP) and hydroxypropylmethyl cellulose (HPMC) are contemplated for use in alcoholic solutions to granulate the therapeutic.

An antifrictional agent may be optionally included in the compositions of the invention to prevent sticking during the composition process. Lubricants may be optionally used as a layer between the therapeutic and the die wall, and these can include but are not limited to: stearic acid including its magnesium and calcium salts, polytetrafluoroethylene (PTFE), liquid paraffin, vegetable oils and waxes. Exemplary soluble lubricants may also be used such as include sodium lauryl sulfate, magnesium lauryl sulfate, polyethylene glycol of various molecular weights, and Carbowax 4000 and 6000.

Glidants that might improve the flow properties of the compound during composition and to aid rearrangement during compression might be optionally added. The glidants may include without limitation starch, talc, pyrogenic silica and hydrated silicoaluminate.

To aid dissolution of the therapeutic agent (e.g. a cannabinoid) into the aqueous environment, a surfactant might be added in certain embodiments as a wetting agent. Surfactants may include, for example and without limitation, anionic detergents such as sodium lauryl sulfate, dioctyl sodium sulfosuccinate and dioctyl sodium sulfonate. Cationic detergents might be optionally used and could include, without limitation, benzalkonium chloride or benzethomium chloride. The list of potential nonionic detergents that could be included in the composition as surfactants are lauromacrogol 400, polyoxyl 40 stearate, polyoxyethylene hydrogenated castor oil 10, 50 and 60, glycerol monostearate, polysorbate 40, 60, 65 and 80, sucrose fatty acid ester, methyl cellulose and carboxymethyl cellulose. When used, these surfactants could be present in the composition of the therapeutic agent either alone or as a mixture in different ratios.

Additives which that potentially enhance uptake of the therapeutic agent include, without limitation, the fatty acids oleic acid, linoleic acid and linolenic acid.

Controlled release composition may be desirable. In certain aspects, the therapeutic agents could be incorporated into an inert matrix that permits release by either diffusion or leaching mechanisms i.e., gums. In some aspects, slowly degenerating matrices may also be incorporated into the composition. Another form of a controlled release of this therapeutic is by a method based on the Oros therapeutic system (Alza Corp.), i.e. the drug is enclosed in a semipermeable membrane which allows water to enter and push drug out through a single small opening due to osmotic effects. Some enteric coatings also have a delayed release effect.

In other aspects, a mix of materials might be used to provide the optimum film coating. Film coating may be carried out, for example, in a pan coater or in a fluidized bed or by compression coating.

### Injectable Compositions

Compositions of the invention may be administered via injection. The route of injection may be subcutaneous, intramuscular, intravenous, intraperitoneal, intracardiac, intraarticular, or intracavernous injection.

The compositions suitable for injectable use optionally include sterile aqueous solutions (where water-soluble) or dispersions and sterile powders for the extemporaneous preparation of sterile injectable solutions or dispersion. Alternatively, the therapeutic agents of the invention are, in certain aspects encapsulated in liposomes and delivered in injectable solutions to assist their transport across cell membrane. Alternatively or in addition such preparations contain constituents of self-assembling pore structures to facilitate transport across the cellular membrane. The carrier, in various aspects, is a solvent or dispersion medium containing, for example, water, ethanol, polyol (for example, glycerol, propylene glycol and liquid polyethylene glycol, and the like), suitable mixtures thereof, and vegetable oils. Proper fluidity is maintained, for example and without limitation, by the use of a coating such as lecithin, by the maintenance of the required particle size in the case of dispersion and by the use of surfactants. Prolonged absorption of the injectable compositions is in certain aspects brought about by the use in the compositions of agents delaying absorption, for example, aluminium monostearate and gelatine.

The invention also provides an injectable sustained release pharmaceutical composition comprising a therapeutically effective pharmaceutical composition according to the invention, and a release retardant. The release retardant may be, for example, aluminium monostearate and gelatine.

Sterile injectable solutions are prepared by incorporating the therapeutic agents in the required amount in an appropriate solvent with various other ingredients enumerated above, as required, followed by filtered sterilisation. Generally, dispersions are prepared by incorporating the various sterilised therapeutic agents into a sterile vehicle that contains the basic dispersion medium and the required other ingredients from those enumerated above. In the case of sterile powders for the preparation of sterile injectable solutions, preparation in certain aspects include without limitation vacuum drying and freeze-drying techniques that yield a powder of the therapeutic agents plus any additional desired ingredient from previously sterile-filtered solution thereof.

### Nasal and Pulmonary Compositions

Compositions of the invention may be administered via nasal or pulmonary delivery.

A wide range of mechanical devices designed for pulmonary delivery of therapeutic agents exist, including but not limited to nebulizers, metered-dose inhalers, and powder inhalers, all of which are familiar to those skilled in the art. Some specific examples of commercially available devices suitable for the practice of this invention are the Ultravent nebulizer, manufactured by Mallinckrodt, Inc., St. Louis, Mo.; the Acorn II nebulizer, manufactured by Marquest Medical Products, Englewood, Colo.; the Ventolin metered dose inhaler, manufactured by Glaxo Inc., Research Triangle Park, N.C.; and the Spinhaler powder inhaler, manufactured by Fisons Corp., Bedford, Mass.

All such devices require the use of compositions suitable for the dispensing of the cannabinoid. Typically, each composition is specific to the type of device employed and may involve the use of an appropriate propellant material, in addition to the usual diluents, adjuvants and/or carriers useful in therapy. Also, the use of liposomes, microcapsules or microspheres, inclusion complexes, or other types of carriers is contemplated.

Compositions suitable for use with a nebulizer, either jet or ultrasonic, will typically comprise the cannabinoid suspended in water or a non-aqueous solvent. The composition may also include a buffer and a simple sugar (e.g., for stabilization and regulation of osmotic pressure). The nebulizer composition may also contain a surfactant, to reduce or prevent surface induced aggregation of the cannabinoid caused by atomization of the solution in forming the aerosol.

Compositions for use with a metered dose inhaler device will generally comprise a finely divided powder containing the cannabinoid suspended in a propellant with the aid of a surfactant. The propellant may be any conventional material employed for this purpose, such as a chlorofluorocarbon, a hydrochlorofluorocarbon, a hydrofluorocarbon, or a hydrocarbon, including trichlorofluoromethane, dichlorodifluoromethane, dichlorotetrafluoroethanol, and 1,1,1,2 tetrafluoroethane, or combinations thereof. Suitable surfactants include sorbitan trioleate and soya lecithin. Oleic acid may also be useful as a surfactant.

Compositions for dispensing from a powder inhaler device will comprise a finely divided dry powder containing the cannabinoid and may also include a bulking agent, such as lactose, sorbitol, sucrose, or mannitol in amounts which facilitate dispersal of the powder from the device, e.g., 50 to 90% by weight of the composition. The cannabinoid should most advantageously be prepared in particulate form with an average particle size of less than 10 microns, most preferably 0.5 to 5 microns, for most effective delivery to the distal lung.

Nasal delivery of a cannabinoid in the treatment methods of the present invention is also contemplated. Nasal delivery allows the passage of the cannabinoid to the blood stream directly after administering the therapeutic product to the nose, without the necessity for deposition of the cannabinoid in the lung. Compositions for nasal delivery include those with dextran or cyclodextran.

### Kits

The invention also provides kits for use in the instant methods. There is therefore provided a kit comprising a cannabinoid for the treatment or prevention of a Neisseria bacterial infection in a subject in need of such treatment or prevention. The invention further provides a kit comprising cannabidiol and/or acids thereof for the treatment or prevention of a Neisseria bacterial infection in a subject in need of such treatment or prevention.

Kits of the invention include one or more containers comprising a cannabinoid as described herein, and instructions for use in accordance with any one of the methods described herein. The kit may further comprise a description of selecting an individual suitable for treatment based on identifying whether that individual has an infection by a Neisseria bacterium. The kit may further comprise a description of administering a cannabinoid as described herein to an individual at risk of developing an infection by a Neisseria bacterium.

Preferably, the cannabinoid is chosen from the list comprising: cannabidiol, cannabinol, cannabigerol, cannabichromene, and Δ⁹-tetrahydrocannabinol. Preferably, the cannabinoid is chosen from the list comprising: cannabidiol, cannabinolic acid. Most preferably, the cannabinoid is cannabidiol.

Preferably the Neisseria bacterial infection to be treated or prevented is caused by Neisseria gonorrhoea or Neisseria meningitidis.

The instructions generally include information as to dosage, dosing schedule, and route of administration for the intended treatment. The containers may be unit doses, bulk packages (e.g. multi0dose packages) or sub-unit doses. Instructions supplied in the kits of the invention are typically written instructions on a label or package insert. The label or package insert indicates that the composition is used for treating, ameliorating and/or preventing an infection by a Neisseria bacterium. Instructions may be provided for practising any of the methods described herein.

### General

Those skilled in the art will appreciate that the invention described herein is susceptible to variations and modifications other than those specifically described. The invention includes all such variation and modifications. The invention also includes all of the steps, features, compositions and therapeutic agents referred to or indicated in the specification, individually or collectively and any and all combinations or any two or more of the steps or features.

Each document, reference, patent application or patent cited in this text is expressly incorporated herein in their entirety by reference, which means that it should be read and considered by the reader as part of this text. That the document, reference, patent application or patent cited in this text is not repeated in this text is merely for reasons of conciseness.

Any manufacturer's instructions, descriptions, product specifications, and product sheets for any products mentioned herein or in any document incorporated by reference herein, are hereby incorporated herein by reference, and may be employed in the practice of the invention.

The present invention is not to be limited in scope by any of the specific embodiments described herein. These embodiments are intended for the purpose of exemplification only. Functionally equivalent products, compositions and methods are clearly within the scope of the invention as described herein.

The invention described herein may include one or more range of values (eg. Size, displacement and field strength etc). A range of values will be understood to include all values within the range, including the values defining the range, and values adjacent to the range which lead to the same or substantially the same outcome as the values immediately adjacent to that value which defines the boundary to the range. Accordingly, unless indicated to the contrary, the numerical parameters set forth in the specification and claims are approximations that may vary depending upon the desired properties sought to be obtained by the present invention. Hence “about 80%” means “about 80%” and also “80%”. At the very least, each numerical parameter should be construed in light of the number of significant digits and ordinary rounding approaches.

Throughout this specification, unless the context requires otherwise, the word “comprise” or variations such as “comprises” or “comprising”, will be understood to imply the inclusion of a stated integer or group of integers but not the exclusion of any other integer or group of integers. It is also noted that in this disclosure and particularly in the claims and/or paragraphs, terms such as “comprises”, “comprised”, “comprising” and the like can have the meaning attributed to it in U.S. Patent law; e.g., they can mean “includes”, “included”, “including”, and the like; and that terms such as “consisting essentially of” and “consists essentially of” have the meaning ascribed to them in U.S. Patent law, e.g., they allow for elements not explicitly recited, but exclude elements that are found in the prior art or that affect a basic or novel characteristic of the invention.

Other definitions for selected terms used herein may be found within the detailed description of the invention and apply throughout. Unless otherwise defined, all other scientific and technical terms used herein have the same meaning as commonly understood to one of ordinary skill in the art to which the invention belongs. The term “active agent” or “therapeutic agent” may mean one active agent or therapeutic agent, or may encompass two or more active agents or therapeutic agents.

The following examples serve to more fully describe the manner of using the above-described invention, as well as to set forth the best modes contemplated for carrying out various aspects of the invention. It is understood that these methods in no way serve to limit the true scope of this invention, but rather are presented for illustrative purposes.

### EXAMPLES

Further features of the present invention are more fully described in the following non-limiting Examples. This description is included solely for the purposes of exemplifying the present invention. It should not be understood as a restriction on the broad description of the invention as set out above.

### Example 1

In this study, to evaluate the spectrum of activity, cannabidiol (CBD) was profiled against Neisseria. Testing was conducted following guidelines recommended by the Clinical and Laboratory Standards Institute (CLSI, 1-4), using vancomycin, meropenem, levofloxacin, and gentamicin as comparators for testing aerobic bacteria.

### Materials and Methods

### Test Agents

Test agents were supplied by Micromyx. Stock solutions of all compounds were prepared on the first day of testing at 101× the highest test concentration using solvents recommended by CLSI. CBD was prepared fresh on each of testing.

Information regarding compound source, lot number, testing concentrations and drug diluent for the comparators and test agents is detailed below:

| | | | Testing Conc Range | |
|---|---|---|---|---|
| Test agent | Supplier | Lot No. | (μg/mL) | Solvent/Diluent |
| CBD | Cerilliant | FE12271801 | 64-0.06 | DMSO/DMSO |
| Vancomycin | Sigma | 080M1341V | 32-0.03 | Water/Water |
| Clindamycin | Sigma | 021M1533 | 32-0.03 | Water/Water |
| Metronidazole | Sigma | 095K0693 | 32-0.03 | DMSO/Water |
| Gentamicin | Sigma | SLBT5354 | 32-0.03 | Water/Water |
| Meropenem | USP | JOK434 | 32-0.03 | Water/Water |
| Levofloxacin | Sigma | BCBF7004V | 32-0.001 | ½ volume of water, |
| | | | | then 0.1M NaOH dropwise |
| | | | | to dissolve/Water |

### Organisms

The test organisms evaluated in this study consisted of clinical isolates from the Micromyx Repository, reference isolates from the American Type Culture Collection (ATCC; Manassas, Va.), isolates from the antibiotic resistance bank from the Centers for Disease Control and Prevention (CDC; Atlanta, Ga.), and isolates from the Network on Antimicrobial Resistance in Staphylococcus aureus (NARSA; BEI Resources, Manassas, Va.). Upon initial receipt at Micromyx, the organisms were sub-cultured onto an appropriate agar medium. Following incubation, colonies were harvested from these plates and cell suspensions prepared and frozen at −80° C. with a cryoprotectant.

Prior to testing, Neisseria was streaked onto Chocolate agar (BD; Lot No. 9228071) and incubated at 35° C. in 5% CO₂ for 24 hr.

### Test Media

For Neisseria, a modified medium described by the ATCC as capable of supporting growth was used. This medium contains 15 g Oxoid Special Peptone, 1 g corn starch (Ward's Science; Rochester, N.Y.; Lot 39-3271), 5 g NaCl (VWR; 57897), 4 g K2HPO4 (Sigma; Lot SLBT7061), 1 g KH2PO4 (SLBC1921V), and 1% IsoVitaleX (BD; Lot 8323954) enrichment per 500 mL.

### Broth Microdilution MIC Testing

MIC values were determined using a broth microdilution procedure described by CLSI (1-4). Automated liquid handlers (Multidrop 384, Labsystems, Helsinki, Finland; Biomek 2000 and Biomek FX, Beckman Coulter, Fullerton, Calif.) were used to conduct serial dilutions and liquid transfers.

To prepare the drug mother plates, which would provide the serial drug dilutions for the replicate daughter plates, the wells of columns 2 through 12 of standard 96-well microdilution plates (Costar 3795) were filled with 150 μl of the designated diluent for each row of drug. The test article and comparator compounds (300 μl at 101× the highest concentration to be tested) were dispensed into the appropriate wells in column 1. The Biomek 2000 was then used to make 2-fold serial dilutions in the mother plates from column 1 through column 11. The wells of column 12 contained no drug and served as the organism growth control wells for the assay.

The daughter plates were loaded with 190 μL per well of the appropriate test medium for the tested organism using the Multidrop 384. The daughter plates were completed on the Biomek FX instrument which transferred 2 μL of drug solution from each well of a mother plate to the corresponding well of each daughter plate in a single step.

A standardized inoculum of each test organism was prepared per CLSI methods (1-4). The inoculum for each organism was dispensed into sterile reservoirs divided by length (Beckman Coulter), and the Biomek 2000 was used to inoculate the plates. Daughter plates were placed on the Biomek 2000 work surface in reverse orientation so that inoculation took place from low to high drug concentration. Plates were inoculated with 10 μL of the inoculum resulting in a final cell density of approximately 5×105 CFU/mL per well.

The plates for testing of aerobic organisms were stacked 3 to 4 high, covered with a sterile lid, and incubated at 35° C. and 5% CO2 for the time periods specified in CLSI (1-4). Plates were incubated for approximately 0 to 24 hr. Following incubation, the microplates were removed from the incubator and viewed from the bottom using a plate viewer. For each of the test media and each drug, an un-inoculated solubility control plate was observed for evidence of drug precipitation and media sterility. The MIC was read and recorded as the lowest concentration of drug that inhibited visible growth of the organism.

### Results

CBD was profiled against Neisseria. The in vitro activity of CBD and the comparators vancomycin, levofloxacin, meropenem and gentamicin against Neisseria is shown is Table 1. Quality control testing of comparator agents against QC organisms were within CLSI published QC ranges. Against the N. meningitidis MMX 0707/ATCC 13090 isolate tested, the CBD MIC value was 0.25 μg/mL.

TABLE 1
In vitro activity of CBD and comparators against
Gram-negative aerobic bacteria

| Bacterial | MMX or | | | | | |
|---|---|---|---|---|---|---|
| Isolates | ATCC No. | VAN | LVX | MEM | GEN | CBD |
| Neisseria | MMX 0683 | 8 | 0.002 | <0.03 | 4 | 1 |
| gonorrhoeae¹ | ATCC 49226 | | | | | |
| N. gonorrhoeae | MMX 6746 | 16 | 4 | 0.06 | 16 | 1 |
| N. gonorrhoeae¹ | MMX 6757 | 32 | 0.015 | <0.03 | 8 | 2 |
| Neisseria | MMX 0707 | >32 | 0.004 | <0.03 | 8 | 0.25 |
| meningitidis | ATCC 13090 | | | | | |

¹For both N. gonorrhoeae isolates MMX 0683/ATCC 49226 and MMX 6757, plates were read at 40 hr as there was no growth at 24 hr
VAN, vancomycin; LVX, levofloxacin; MEM, meropenem; GEN, gentamicin
*plate read at 24 hr
**plate read at 48 hr

### Example 2

In this study, the in vitro spectrum of activity of CBD was determined against 30 isolates comprised of reference strains from the WHO, isolates from the CDC antibiotic resistance bank, and clinical isolates from the Micromyx (MMX) repository, with different resistance phenotypes. Assays were conducted following guidelines recommended by the Clinical and Laboratory Standards Institute (CLSI; 1, 2) with the exception that broth microdilution was used instead of agar dilution. Comparator antibiotics used in this study were ceftriaxone, ciprofloxacin, and azithromycin.

### Materials and Methods

### Test Compounds and Comparators

The test agent, CBD, and the comparator compounds, azithromycin, ceftriaxone, and ciprofloxacin were supplied by Micromyx, and stored at the appropriate temperature until use. Stock solutions of all compounds were prepared on the day of testing using solvents recommended by CLSI. Stock solutions of all compounds were made at 101× the final testing concentration. CBD was supplied by Cerilliant (Round Rock, Tex.) as a 1 mg/mL methanolic solution which was stored at −20° C. after receipt. Prior to testing, using a 5 mL aliquot of the CBD methanolic solution, the methanol was dried off and the residual CBD was resuspended in 774 μL DMSO resulting in a 6.464 mg/mL solution.

Information regarding compound source, lot number, testing concentrations and drug diluent for the comparator and test agent is detailed below:

| | | | Testing | |
|---|---|---|---|---|
| | | Catalog/ | Concentration | |
| Test agent | Supplier | Lot No. | Range (μg/mL) | Solvent/Diluent |
| CBD | Cerilliant | C-045/ | 64-0.06 | DMSO/DMSO |
| | (Sigma) | Fe12271801 | | |
| Azithromycin | USP | 1046056/ | 8-0.008 | Water/Water |
| | | RO43PO | | |
| Ceftriaxone | USP | 1098184/ | 8-0.008 | Water/Water |
| | | R07420 | | |
| Ciprofloxacin | USP | 1134335/ | 8-0.008 | Water/Water |
| | | R05170 | | |

### Test Isolates

The test organisms evaluated in this study consisted of Neisseria gonorrhoeae isolates from the Micromyx repository and reference isolates from the American Type Culture Collection (ATCC; Manassas, Va.), Centers for Disease Control and Prevention (CDC; Atlanta, Ga.) or the National Collection of Type Cultures (NCTC; Public Health England, Salisbury, UK). Upon initial receipt at Micromyx, the organisms were sub-cultured onto an appropriate agar medium.

Following incubation, colonies were harvested from these plates and cell suspensions prepared and frozen at −80° C. with a cryoprotectant. Prior to testing, N. gonorrhoeae isolates were streaked onto Chocolate Agar [Becton Dickinson, (BD), Sparks, Md.; Lot No. 922807]; Staphylococcus aureus ATCC 29213 tested for quality control (QC) purposes was streaked onto Trypticase Soy Agar with 5% Sheep Blood (TSA II; BD; Lot No. 9219399).

Test Media

For N. gonorrhoeae, a modified medium described by the ATCC as capable of supporting the growth of N. gonorrhoeae (Neisseria peptone medium) was used for the broth microdilution MIC assay. This medium contained 15 g Oxoid Special Peptone (Lot No. 1280296; Oxoid, Hampshire, UK), 1 g corn starch (Lot No. AD-13344-14; Ward's Science; Rochester, N.Y.), 5 g NaCl (Lot No. 57897; VWR, Radnor, Pa.), 4 g K2HPO4 (Lot No. SLBT7061; Sigma), and 1 g KH2PO4 (Lot No. SLBC1921V; Sigma). After autoclaving, the medium was centrifuged at 5,000×g for 10 min, the supernatant was passed through a 0.45 μm filter, and IsoVitaleX supplement (Lot No. 8323954; BD) was added at 1% (v/v).

Cation-adjusted Mueller Hinton broth (CAMHB; BD; Lot No. 9019592) was used for MIC testing of S. aureus ATCC 29213 for QC purposes.

### Broth Microdilution MIC Assay

MIC values were determined using a broth microdilution procedure described by CLSI (1, 2). Automated liquid handlers (Multidrop 384, Labsystems, Helsinki, Finland; Biomek 2000 and Biomek FX, Beckman Coulter, Fullerton Calif.) were used to conduct serial dilutions and liquid transfers.

To prepare the drug mother plates, which would provide the serial drug dilutions for the replicate daughter plates, the wells of columns 2 through 12 of standard 96-well microdilution plates (Costar 3795) were filled with 150 μl of the designated diluent for each row of drug. The test articles and comparator compounds (300 μl at 100× the highest concentration to be tested) were dispensed into the appropriate wells in column 1. The Biomek 2000 was then used to make 2-fold serial dilutions in the mother plates from column 1 through column 11. The wells of column 12 contained no drug and served as the organism growth control wells for the assay.

The daughter plates were loaded with 190 μL per well of the appropriate test medium for the tested organism using the Multidrop 384. The daughter plates were completed on the Biomek FX instrument which transferred 2 μL of drug solution from each well of a mother plate to the corresponding well of each daughter plate in a single step. Daughter plates for the testing of anaerobes were allowed to pre-reduce in the Bactron II anaerobe chamber for 2 hr prior to inoculation.

A standardized inoculum of each test organism was prepared per CLSI methods (1, 2). The inoculum for each organism was dispensed into sterile reservoirs divided by length (Beckman Coulter), and the Biomek 2000 was used to inoculate the plates. Daughter plates were placed on the Biomek 2000 work surface in reverse orientation so that inoculation took place from low to high drug concentration. Anaerobic organisms were inoculated by hand in the Bactron II anaerobic chamber. Bacterial plates were then inoculated with 10 μL of the inoculum resulting in a final cell density of approximately 5×105 CFU/mL.

Plates were stacked 3 to 4 high, covered with a lid on the top plate and placed in plastic bags. N. gonorrhoeae plates were incubated at 35° C. in 5% CO2 for 24 hr, and the S. aureus plate was incubated for 16 to 20 hr at 35° C. at ambient atmosphere. Following incubation, the microplates were removed from the incubator and viewed from the bottom using a plate viewer. For each of the test media and each drug, an un-inoculated solubility control plate was observed for evidence of drug precipitation. The MIC was read and recorded as the lowest concentration of drug that inhibited visible growth of the organism.

### Results

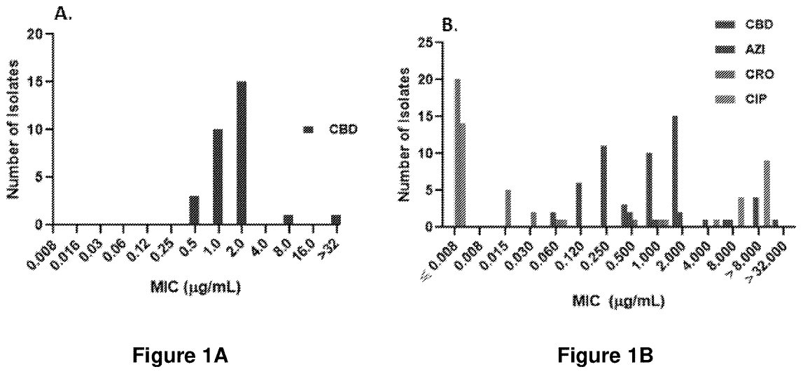

The activity of CBD and the comparators azithromycin, ceftriaxone and ciprofloxacin against 30 isolates of Neisseria gonorrhoeae is shown in Table 2. A summary of this activity, with MIC distributions can be found in Table 4, and the activity is presented graphically in FIG. 1. All quality control testing of the comparators against Staphylococcus aureus ATCC 29213 were within CLSI QC ranges and QC testing against N. gonorrhoeae ATCC 49226 was within range for azithromycin and ciprofloxacin and below the range for ceftriaxone. However, these ranges are for agar dilution testing and as ceftriaxone tested in QC with S. aureus ATCC 29213 the panels as used during the study were validated. Of note, precipitation of CBD was seen at 8 μg/mL and above for the Neisseria test medium and CAMHB.

Included in this panel were isolates of N. gonorrhoeae from the Micromyx repository and reference isolates from the ATCC, CDC or the NCTC, with different resistance phenotypes. As shown in Table 3 and FIG. 1, the MIC50 for CBD against this panel of N. gonorrhoeae isolates was 2 μg/mL, the MIC90 was 2 μg/mL, and the MIC range was 0.5 to >32 μg/mL. This activity was not affected by the resistance phenotype of the isolate. Against this collection of N. gonorrhoeae, azithromycin had MIC50/90 values of 0.25/>8 μg/mL, and a range of 0.06 to >8 μg/mL. MIC50/90 values for ceftriaxone and ciprofloxacin against these isolates were ≤0.008/0.03 and 0.06/>8 μg/mL, respectively, with MIC ranges of ≤0.008 to 1 and ≤0.008 to >8 μg/mL, respectively.

In this study, CBD was evaluated for activity against 30 isolates of N. gonorrhoeae with different resistance phenotypes, in comparison to azithromycin, ceftriaxone and ciprofloxacin. Overall, CBD demonstrated MIC50/90 values of 2/2 μg/mL against this panel, and this activity was observed to be independent of the resistance phenotype of the tested isolate.

TABLE 2
In vitro activity of CBD and comparators against 30 isolates of Neisseria gonorrhoeae

| | | MIC (μg/mL) | | | |
|---|---|---|---|---|---|
| Number | Type | CBD | AZI | CRO | CIP |

Staphylococcus aureus

| | | | | | |
|---|---|---|---|---|---|
| ATCC 29213 | QC | 0.5 | 0.5 (0.5-2)¹ | 2 (1-8)¹ | 0.25 (0.12-0.5)¹ |

Neisseria gonorrhoeae

| | | | | | |
|---|---|---|---|---|---|
| ATCC 49226 | QC | 2 | 0.25 (0.25-1)¹ | ≤0.008 | ≤0.008 |
| | | | | (0.016- | (0.008- |
| | | | | 0.004)¹ | 0.001)¹ |
| NCTC 13477 | WHO F; CIP-S, AZI MIC 0.12 | 0.5 | 0.06 | ≤0.008 | ≤0.008 |
| NCTC 13478 | WHOG; CIP-I, AZI MIC 0.25 | 2 | 0.25 | ≤0.008 | 0.06 |
| NCTC 13479 | WHO K; CIP-R, AZI MIC 0.25 | 2 | 0.25 | 0.03 | >8 |
| NCTC 13480 | WHO L; CIP-R, AZI MIC 0.5 | 0.5 | 0.25 | 0.06 | >8 |
| NCTC 13481 | WHO M; CIP-R, AZI MIC 0.25 | 1 | 0.12 | ≤0.008 | 1 |
| NCTC 13482 | WHO N; CIP-R, AZI MIC 0.25 | 2 | 0.12 | ≤0.008 | 4 |
| NCTC 13483 | WHO O; CIP-S, AZI MIC 0.25 | 2 | 0.25 | ≤0.008 | ≤0.008 |
| NCTC 13484 | WHO P; CIP-S, AZI MIC 4 | >32² | 4 | ≤0.008 | ≤0.008 |
| NCTC 13817 | WHO U; CIP-S, AZI MIC 4 | 1 | 2 | ≤0.008 | ≤0.008 |
| NCTC 13818 | WHO V; CIP-R, AZI MIC >256 | 1 | >8 | ≤0.008 | >8 |
| NCTC 13819 | WHO W; CIP-R, AZI MIC 0.5 | 1 | 0.25 | 0.015 | >8 |
| NCTC 13820 | WHO X; CRO-NS, CIP-R,AZI | 2 | 0.25 | 1 | >8 |
| | MIC 0.5 | | | | |
| NCTC 13821 | WHO Y; CIP-R, AZI MIC 1 | 1 | 0.5 | 0.5 | >8 |
| MMX 10409 | CIP-R | 2 | 0.25 | 0.015 | >8 |
| MMX 10410 | CIP-S | 2 | 0.12 | ≤0.008 | ≤0.008 |
| MMX 10447 | CIP-S | 1 | 0.06 | ≤0.008 | ≤0.008 |
| MMX 10450 | CIP-S | 0.5 | 0.12 | ≤0.008 | ≤0.008 |
| MMX 10452 | CIP-R | 2 | 0.12 | ≤0.008 | >8 |
| MMX 10454 | CIP-R | 1 | 0.12 | 0.015 | 8 |
| MMX 6921 | CIP-R | 1 | 8 | ≤0.008 | 8 |
| MMX 6938 | CIP-S | 2 | 0.25 | ≤0.008 | ≤0.008 |
| CDCO165 | CIP-R,AZI MIC 1 | 8 | 1 | 0.015 | >8 |
| CDC0166 | CIP-R, AZI MIC 1 | 2 | 0.5 | 0.015 | 8 |
| CDCO167 | CIP-S,AZI MIC 8 | 1 | >8 | ≤0.008 | ≤0.008 |
| CDC 0172 | CIP-R,AZI MIC 0.5 | 2 | 0.25 | ≤0.008 | 8 |
| CDC 0177 | CIP-S,AZI MIC 2 | 2 | 2 | ≤0.008 | ≤0.008 |
| CDC 0175 | CIP-S,AZI MIC16 | 2 | >8 | ≤0.008 | ≤0.008 |
| CDC 0181 | CIP-S,AZI MIC 256 | 1 | >8 | ≤0.008 | ≤0.008 |
| CDC 0194 | CRO-NS, CIP-S, AZI MIC 0.5 | 2 | 0.25 | 0.03 | ≤0.008 |

¹ CLSI QC ranges shown in parentheses;
² growth above 32 ug/mL was unable to be determined due to CBD precipitation.
AZI, azithromycin; CRO, ceftriaxone; CIP, ciprofloxacin. S, susceptible; R, resistant; NS, non-susceptible

TABLE 3
Summary of Activity of CBD and Comparators against 30 Isolates of N. gonorrhoeae

| | MIC value (μg/mL) | | | | | | | | | | | | | |
|---|---|---|---|---|---|---|---|---|---|---|---|---|---|---|
| Drug | <0.008 | 0.008 | 0.015 | 0.03 | 0.06 | 0.12 | 0.25 | 0.5 | 1 | 2 | 4 | 8 | >8 | >32 |
| CBD | | | | | | | | 3 | 10 | 15 | | 1 | | 1 |
| AZI | | | | | 2 | 6 | 11 | 2 | 1 | 2 | 1 | 1 | 4 | |
| CRO | 20 | | 5 | 2 | 1 | | | 1 | 1 | | | | | |
| CIP | 14 | | | | 1 | | | | 1 | | 1 | 4 | 9 | |

AZI, azithromycin; CRO, ceftriaxone; CIP, ciprofloxacin

TABLE 4
Summary of Activity of CBD and Comparators
against 30 Isolates of N. gonorrhoeae

| | MIC Range | MIC Mode | MIC50 | MIC90 |
|---|---|---|---|---|
| Drug | (μg/mL) | (μg/mL) | (μg/mL) | (μg/mL) |
| CBD | 0.5 −> 32 | 2 | 2 | 2 |
| AZI | 0.06 −> 8 | 0.25 | 0.25 | >8 |
| CRO | ≤0.008 − 1 | ≤0.008 | ≤0.008 | 0.03 |
| CIP | ≤0.008 −> 8 | ≤0.008 | 0.06 | >8 |

AZI, azithromycin; CRO, ceftriaxone; CIP, ciprofloxacin

### Example 3

The MIC of Neisseria gonorrhoea was established using an agar dilution MIC assay.

### Materials and Methods

### Test Compounds and Comparators

Test drugs were provided by Micromyx. All stock solutions were allowed to stand for at least 1 hr prior to use to auto-sterilize. Leftover stock solutions of comparator drugs were aliquoted and stored at −80° C. Suppliers, catalog/lot numbers, solvents, stock concentrations and testing ranges were as follows.

| | | | Testing Cone Range | |
|---|---|---|---|---|
| Test agent | Supplier | Catalog/Lot No. | (ug/mL) | Solvent/Diluent |
| CBD | Cerilliant (Sigma) | C-045/ | 32-0.03 | DMSO/DMSO |
| Ciprofloxacin | USP | 1134335/R05170 | 8-0.005 | Water/Water |
| Ceftriaxone | Sigma | C5793/077K0548 | 8-0.005 | Water/Water |

### Test Isolates

Test organisms were reference strains from the American Type Culture Collection (ATCC; Manassas, Va.), isolates from WHO, isolates from the CDC antibiotic resistance bank, and clinical isolates from the Micromyx (MMX) repository. Upon receipt at Micromyx, the isolates were streaked under suitable conditions onto agar medium appropriate to each organism and were incubated for 24-48 hr at 35° C. Colonies harvested from these growth plates were resuspended in the appropriate medium containing a cryoprotectant. Aliquots of each suspension were then frozen at −80° C.

Prior to testing, the isolates were sub-cultured onto Chocolate agar (Becton Dickenson [BD]/BBL; Sparks, Md., Cat. No. 221267, Lot No. 9346275) and placed at 35° C. in a CO2 incubator for 24 hrs.

### Test Medium

For N. gonorrhoeae, GC agar medium (BD, Cat. No. 228950; Lot No. 4191863) supplemented with 1% IsoVitalex (BD, Cat. 211876; Lot No. 9203982) was used for the MIC assay.

### Agar Dilution MIC Methodology

MIC values were determined using the agar dilution method (1, 2). All serial dilutions and liquid handling were performed by hand using sterile pipettes. Ciprofloxacin and ceftriaxone were prepared as stock solutions at 40× in the appropriate solvent and diluted according to CLSI guidelines. Cannabidiol was prepared as a stock solution at 100× the final concentration in the appropriate solvent and diluted according to CLSI guidelines.

Each test agent was mixed with molten (50-55° C.) GC media agar supplemented with 1% IsoVitalex. Ciprofloxacin and ceftriaxone were added to the medium in a ratio of 2 mL 10× test agent to 18 mL agar. All other test agents were added to the medium in a ratio of 0.2 mL 100× test agent to 19.8 mL agar. Once test agents were added to the agar in a sterile tube, they were mixed gently and then poured into a sterile 110×15 mm petri dish. Plates were allowed to solidify at room temperature and placed in a laminar air flow hood with the covers off to remove condensed moisture on the agar surface.

Next, each isolate was suspended to the equivalent of a 0.5 McFarland standard in saline using a Siemens Microscan turbidity meter and diluted 1:10. Each bacterial cell suspension was then transferred to wells in a stainless-steel replicator block. The prongs on the replicator deliver approximately 1-2 μl of inoculum to an agar surface. The resulting inoculum spots contained approximately 10⁴ cells/spot.

Each agar plate containing either test compound or no drug (control) was stamped with the replicator. All plates were placed with the agar surface up to allow for the inoculum to soak into the agar. The plates were inverted and incubated at 35° C. for 24 hrs in a CO2 incubator and finally inspected for growth. The Minimum Inhibitory Concentration (MIC) was defined as the lowest test agent concentration that substantially inhibited bacterial growth on the agar surface.

### Test Isolates

The test organisms evaluated in this study consisted of Neisseria gonorrhoeae isolates from the Micromyx repository and reference isolates from the American Type Culture Collection (ATCC; Manassas, Va.), Centers for Disease Control and Prevention (CDC; Atlanta, Ga.) or the National Collection of Type Cultures (NCTC; Public Health England, Salisbury, UK). Upon initial receipt at Micromyx, the organisms were sub-cultured onto an appropriate agar medium.

Following incubation, colonies were harvested from these plates and cell suspensions prepared and frozen at −80° C. with a cryoprotectant. Prior to testing, N. gonorrhoeae isolates were streaked onto Chocolate Agar [Becton Dickinson, (BD), Sparks, Md.; Lot No. 922807]; Staphylococcus aureus ATCC 29213 tested for quality control (QC) purposes was streaked onto Trypticase Soy Agar with 5% Sheep Blood (TSA II; BD; Lot No. 9219399).

### Results

Similar MIC values were obtained for 25 isolates when assayed under agar dilution assay conditions as were obtained for the microbroth dilution in Example 2 (Table 5): MIC50/MIC90 for CBD was 1/2 μgmL-1, ciprofloxacin 4/>8 μg mL-1 and ceftriaxone 0.015/0.25 μg mL⁻¹ (Table 6).

TABLE 5
Minimum inhibitory concentrations (MICs) of CBD (μg mL⁻¹) and comparators against
30 isolates of Neisseria gonorrhoeae by broth microdilution assay, and 25 isolates by agar
dilution assay (n = 1).

| | | MIC (fig mL) | | | | | | |
|---|---|---|---|---|---|---|---|---|
| | | Broth Micredilution | | | | Agar Dilution | | |
| Strain | Type | CBD | Azithromycin | Ceftriaxone | Ciprofloxacin | CBD | Ceftriaxone | Ciprofloxacin |

Staphylococcus aureus

| | | | | | |
|---|---|---|---|---|---|
| ATCC 29213 | QC | 0.5 | 0.5 | 2 | 0.25 |
| | | | (0.5-2)¹ | (1-8)¹ | (0.12-0.5)¹ |

Neisseria gonorrhoeas

| | | | | | | | | |
|---|---|---|---|---|---|---|---|---|
| ATCC 49226 | QC | 2 | 0.25 | £0.008 | <0.008 | 2 | 0.015 | 0.004 |
| | | | (0.25-2)¹ | (0.016- | (0.008- | | (0.004- | (0.001- |
| | | | | 0.004)¹ | 0.001)¹ | | 0.015) | 0.008) |
| NCTC 13477 | WHO F; CIP-S: AZI ME 0.12 | 0.5 | 0.06 | ≤0.008 | <0.008 | 1 | <0.004 | 0.004 |
| NCTC 13478 | WHO G CIP-I: AZI MIC 0.25 | 2 | 0.25 | ≤0.008 | 0.06 | 1 | 0.015 | 0.12 |
| NCTC 13479 | WHO K; CIP-R: AZI MIC 0.25 | 2 | 0.25 | 0.03 | >8 | 0.5 | 0.015 | 4 |
| NCTC 13480 | WHO L: CIP-R: AZI MIC 0.5 | 0.5 | 0.25 | 0.06 | >8 | 2 | 0.25 | >8 |
| NCTC 13481 | WHO M; CIP-R: AZI ME 0.25 | 1 | 0.12 | ≤0.008 | 1 | 1 | 0.008 | 2 |
| NCTC 13482 | WHO N; CIP-R: AZI MIC 0.25 | 2 | 0.12 | ≤0.008 | 4 | 1 | 0.008 | 4 |
| NCTC 13483 | WHO O: CIP-S: AZI MIC 0.25 | 2 | 0.25 | ≤0.008 | ≤0.008 | 1 | 0.015 | 0.015 |
| NCTC 13484 | WHO P; CIP-S: AZI MIC 4 | >32² | 4 | ≤0.008 | ≤0.008 | 7 | 0.00:8 | 0.008 |
| NCTC 13817 | WHO U; CIP-S: AZI MIC 4 | 1 | 2 | ≤0.008 | ≤0.008 | 1 | 0.002 | 0.004 |
| NCTC 13818 | WHO V; CIP-R AZI MIC >256 | 1 | >8 | ≤0.008 | >8 | 1 | 0.015 | >8 |
| NCTC 13819 | WHO W; CIP-R, AZI MIC 0.5 | 1 | 0.25 | 0.015 | >8 | 1 | 0.12 | >8 |
| NCTC 13820 | WHO X; CRO-NS CIP-R: AZI | 2 | 0.25 | 1 | >8 | 1 | 2 | >8 |
| | MIC 0.5 | | | | | | | |
| NCTC 13821 | WHO Y; CIP-R: AZI MIC 1 | 1 | 0.5 | 0.5 | >8 | NG | NG | NG |
| MMX 10409 | CIP-R | 2 | 0.25 | 0.015 | >8 | 1 | 0.06 | >8 |
| MMX 10410 | CIP-S | 2 | 0.12 | ≤0.008 | ≤0.008 | | | |
| MMX 10447 | CIP-S | 1 | 0.06 | ≤0.008 | ≤0.008 | | | |
| MMX 10450 | CIP-S | 0.5 | 0.12 | ≤0.008 | ≤0.008 | | | |
| MMX 10452 | CIP-R | 2 | 0.12 | ≤0.008 | >8 | | | |
| MMX 10454 | CIP-R | 1 | 0.12 | 0.015 | 8 | | | |
| MMX 6921 | CIP-R | 1 | 8 | ≤0.008 | 8 | | | |
| MMX 6938 | CIP-S | 2 | 0.25 | ≤0.008 | ≤0.008 | | | |
| CDC0165 | CIP-R: AZI MIC 1 | 8 | 1 | 0.015 | >8 | 2 | 0.06 | >8 |
| CDC0166 | CIP-R- AZI MIC 1 | 2 | 0.5 | 0.015 | 8 | 9 | 0.06 | >8 |
| CDC0167 | CIP-S: .AZI MIC 8 | 1 | >8 | ≤0.008 | ≤0.008 | 1 | 0.004 | 0.004 |
| CDC 0172 | CIP-R: AZI MIC 0.5 | 2 | 0.25 | ≤0.008 | 8 | 7 | 0.06 | >8 |
| CDCO174 | NA | | | | | 2 | 0.06 | >8 |
| CDC 0175 | CIP-S: AZI MIO 16 | 2 | >8 | ≤0.008 | ≤0.008 | 1 | 0.008 | 0.004 |
| CDC 0177 | CIP-S: AZI MIO 2 | 2 | 2 | ≤0.008 | ≤0.008 | 1 | 0.015 | 0.015 |
| CDC 0178 | NA | | | | | 2 | 0.06 | >8 |
| CDC 0180 | NA | | | | | 7 | 0.03 | >8 |
| CDC 0181 | CIP-S: AZI MIC 255 | 1 | >8 | ≤0.008 | ≤0.008 | 0.5 | 0.008 | 0.015 |
| C DC 0194 | CRO-NS, CIP-S: AZI MIC 0.5 | 2 | 0.25 | 0.03 | ≤0.008 | 1 | 0.25 | 0.004 |

¹CLSI QC ranges shown in parentheses;
²growth above 32 μg mL-1 was unable to be determined due to CBD precipitation.
AZI, azithromycin; CRO, ceftriaxone; CIP, ciprofloxacin; S, susceptible; R, resistant; NS, non-susceptible; NG, no growth

TABLE 6

| MIC50/MIC90 for CBD | | | | | | | | | | | | | |
|---|---|---|---|---|---|---|---|---|---|---|---|---|---|
| Antibiotic | ≤0.004 | 0.08 | 0.015 | 0.03 | 0.06 | 0.12 | 0.25 | 0.5 | 1 | 2 | 4 | 8 | >8 |

Neisseria gonorrhea MIC distribution (μg mL⁻¹) broth dilution assay (30 isolates)

| | | | | | | | | | | | | | |
|---|---|---|---|---|---|---|---|---|---|---|---|---|---|
| Ceftriaxone | 20 | 0 | 5 | 2 | 1 | 0 | 0 | 1 | 1 | 0 | 0 | 0 | 0 |
| Azithromycin | 0 | 0 | 0 | 0 | 2 | 6 | 11 | 2 | 1 | 2 | 1 | 1 | 4 |
| Ciprofloxacin | 14 | 0 | 0 | 0 | 1 | 0 | 0 | 0 | 1 | 0 | 1 | 4 | 9 |
| Cannabidiol | 0 | 0 | 0 | 0 | 0 | 0 | 0 | 3 | 10 | 15 | 0 | 1 | 1 |

Neisseria gonorrheae MIC distribution (μg mL⁻¹) agar dilution assay (25 isolates)

| | | | | | | | | | | | | | |
|---|---|---|---|---|---|---|---|---|---|---|---|---|---|
| Ceftriaxone | 3 | 5 | 6 | 1 | 6 | 1 | 2 | 0 | 0 | 1 | 0 | 0 | 0 |
| Ciprofloxacin | 6 | 1 | 3 | 0 | 0 | 1 | 0 | 0 | 0 | 1 | 2 | | 11 |
| Cannabidiol | 0 | 0 | 0 | 0 | 0 | 0 | 0 | 2 | 14 | 9 | 0 | 0 | 0 |

### REFERENCES

1. Clinical and Laboratory Standards Institute (CLSI). Methods for Dilution Antimicrobial Susceptibility Tests for Bacteria That Grow Aerobically; Approved Standard—Eleventh Edition. CLSI document M07-A11. CLSI, 950 West Valley Road, Suite 2500, Wayne, Pa. 19087 USA, 2018.
2. CLSI. Performance Standards for Antimicrobial Susceptibility Testing; 29th ed. CLSI supplement M100. CLSI, 950 West Valley Road, Suite 2500, Wayne, Pa. 19087 USA, 2019.
3. CLSI. Methods for Antimicrobial Susceptibility Testing of Anaerobic Bacteria; Approved Standard—Ninth Edition. CLSI document M11-A9. CLSI, 950 West Valley Road, Suite 2500, Wayne, Pa. 19087 USA, 2018.
4. CLSI. Methods for Antimicrobial Dilution and Disk Susceptibility Testing of Infrequently Isolated or Fastidious Bacteria; 3rd ed. CLSI guideline M45. CLSI, 940 West Valley Road, Suite 2500, Wayne, Pa. 19087 USA, 2016.

1. A composition comprising a cannabinoid for use in the treatment or prevention of an infection by a Neisseria bacterium.

2. (canceled)

3. (canceled)

4. (canceled)

5. (canceled)

6. (canceled)

7. (canceled)

8. (canceled)

9. (canceled)
